(19) United States
(12) Patent Application Publication
(10) Pub. No.: US 2009/0104668 A1
(43) Pub. Date: Apr. 23, 2009

(54) Method for Producing Biopterins Using Tetrahydrobiopterin Biosynthesis Enzyme

(75) Inventors: Ichiro Shimizu, Osaka (JP); Yasuhiro Ikenaka, Hyogo (JP)

Correspondence Address: BIRCH STEWART KOLASCH & BIRCH, PO BOX 747, FALLS CHURCH, VA, 22040-0747, US

(73) Assignee: Kaneka Corporation, Osaka-shi (JP)

(21) Appl. No.: 11/795,322

(22) PCT Filed: Feb. 7, 2006
(86) PCT No.: PCT/JP2006/002087

§ 371 Date: Jul. 16, 2007

(30) Foreign Application Priority Data
Feb. 9, 2005 (JP) 2005-032736

(51) Int. Cl. C12P 17/18; C12N 9/00; C07H 21/04; C12N 15/64; C12N 1/21

(52) U.S. Cl. 435/119; 435/183; 536/23.1; 435/320.1; 435/252.33; 435/252.3

(57) ABSTRACT

Biopterins are useful compounds utilized in pharmaceutical agents or functional foods. The presence of sepiapterin reductase (SPR) involved in the biosynthesis of biopterins has not been confirmed so far in microorganisms except for a few microorganisms such as blue-green algae. For efficiently producing biopterins using microorganisms, it has been demanded to obtain and use SPR genes derived from microorganisms. The present inventors have found that when Saccharomyces cerevisiae or Escherichia coli is transformed with a YIR035C gene from Saccharomyces cerevisiae or a yueD gene from Bacillus subtilis, the transformed microorganism secretes biopterins into a culture solution. Based on this finding, the present invention provides a polypeptide, DNA encoding the polypeptide, a recombinant vector comprising the DNA, and a transformant obtained by transformation with the vector, which are useful in biopterin production using microorganisms. Moreover, the present invention provides a method for efficiently producing biopterins using the transformant.

### TECHNICAL FIELD

The present invention relates to a microorganism-derived gene encoding an enzyme involved in tetrahydrobiopterin biosynthesis, a transformed cell introducing the gene therein, and a method for producing biopterins using the transformed cell.

### CROSS-REFERENCES TO RELATED APPLICATIONS

All the disclosed contents of Japanese Patent Application No. 2005-032736 (issued on Feb. 9, 2005) including specification, claims, drawings, and abstract are incorporated into the present application by reference to all these disclosed contents.

### BACKGROUND ART

In the present specification, tetrahydrobiopterin refers to L-erythro-5,6,7,8-tetrahydrobiopterin (hereinafter, abbreviated to BH4), and the tetrahydrobiopterin and its oxidized forms L-erythro-7,8-dihydrobiopterin (hereinafter, abbreviated to BH2) and L-erythro-biopterin (hereinafter, abbreviated to biopterin) are collectively referred to as biopterins.

It has been known as to biopterins produced by a method of the present invention that a biopterin oxidized form was isolated for the first time as a trypanosome growth factor from human urine by Patterson et al., in 1955 (see Non-Patent Document 1) and is also present in relatively large amounts in a variety of organs, the skins of certain species of reptiles, amphibians, and fishes, the eyes of drosophila, and so on.

Chemical synthetic methods and biological methods have been known as conventional methods for producing biopterins.

Organic synthesis methods from sugars such as rhamnose have been used as examples of the chemical synthetic methods and have been used industrially in the production of BH4 as a pharmaceutical agent. However, current production methods using chemical synthesis have problems such as expensive substrates used such as rhamnose as well as complicated reaction procedures and inevitable use of hard-to-handle agents.

On the other hand, methods involving extraction from the organisms as well as methods using microorganisms (see Patent Documents 1 and 2) or biosynthesis enzymes (see Patent Document 3) have been studied as the biological methods. However, all of these methods are significantly insufficient in productivity and have not been put into practical use so far.

Three biosynthesis enzymes, GTP cyclohydrolase I (hereinafter, abbreviated to GCHI), pyruvoyltetrahydropterin synthase (hereinafter, abbreviated to PTPS), and sepiapterin reductase (hereinafter, abbreviated to SPR), are generally involved in BH4 biosynthesis with guanosine triphosphate (hereinafter, abbreviated to GTP) as a substrate. If these enzymes can be expressed in large amounts and the GTP as a substrate can be supplied in large amounts, BH4 may be produced in large amounts. PTPS and SPR genes derived from microorganisms have not been known except for a few microorganisms such as blue-green algae. The microorganisms do not produce BH4 itself. Therefore, genes derived from organisms other than microorganisms, such as mammals have been required to be used as these two biosynthesis enzyme genes.

Recently, a considerable level of productivity has been achieved in Escherichia coli using gene recombination approaches (see Patent Document 4).

### Patent Document 1: Japanese Patent Publication No. 5-33989

### Patent Document 2: Japanese Patent Publication No. 5-33990

### Patent Document 3: Japanese Patent Laid-Open No. 4-82888

Patent Document 4: International Publication No. WO2002-018587
Non-Patent Document 1: J. Am. Chem. Soc., 77, 3167-3168 (1955)

### DISCLOSURE OF THE INVENTION

### Problems to be Solved by the Invention

However, the technique disclosed in Patent Document 4 may result in instable expression due to combined use of several mammal-derived biosynthesis enzyme genes and the like.

The applications and utilization of biopterins have been expanded increasingly by virtue of their actions as pharmaceutical agents as well as their effects that can be expected as functional foods. Thus, it has been demanded to efficiently produce those having safe quality in large amounts. Particularly, for using the biopterins as foods such as functional foods, a production system thereof is important which is capable of stably supplying those having safe quality. Production using microorganisms may be excellent in these regards. However, current techniques use enzyme genes derived from mammals such as rats as biosynthesis enzyme genes, resulting in the instable expression of the biosynthesis enzymes or the insufficient stability of the genes themselves in production using microorganisms such as Escherichia coli and Saccharomyces cerevisiae. Therefore, these enzyme genes derived from mammals seem to be difficult to use efficiently and stably. Thus, it has been demanded to obtain and use biosynthesis enzyme gene derived from microorganisms.

### Means for Solving the Problems

The present inventors have widely searched, regarding SPR, one of biosynthesis enzyme genes of biopterins, for sequences highly homologous to human or mouse SPR enzyme protein sequences in the database of known genes from microorganisms. As a result, a Saccharomyces cerevisiae YIR035C gene has been found as a relatively highly homologous sequence (28% to the human SPR gene, 26% to the mouse SPR gene), though the presence of biosynthesis pathways of biopterins in Saccharomyces cerevisiae has not been known. SPR activities have been unconfirmed in proteins expressed from the gene. Therefore, the present inventors have conducted studies on its effect. Moreover, the present inventors have also conducted studies on the effect of a yueD gene derived from Bacillus subtilis, one of SPR-like sequences, for which SPR activities have been unconfirmed.

As a result, surprisingly, it has been found that when Saccharomyces cerevisiae YIR035C and Bacillus subtilis yueD DNAs were separately introduced into Saccharomyces cerevisiae or Escherichia coli, the Saccharomyces cerevisiae or Escherichia coli secretes into a culture solution, biopterins that are not usually produced by them. It has further been found that BH4 biosynthesis enzyme genes such as GCHI and PTPS derived from a variety of organisms are introduced simultaneously therewith into the microorganisms to thereby significantly increase the production of biopterins.

Accordingly, the present invention encompasses the following one or several characteristics:

(1) A polypeptide of the following (A) or (B):
(A) a polypeptide comprising the amino acid sequence of SEQ ID NO: 1 or 2; and
(B) a polypeptide having an amino acid sequence derived from the amino acid sequence of the polypeptide (A) with the substitution, deletion, and/or addition of one or several amino acids and having a sepiapterin reductase activity.
(2) Isolated DNA of the following (a) or (b):
(a) DNA comprising the nucleotide sequence of SEQ ID NO: 3 or 4; and
(b) DNA hybridizing under stringent conditions to DNA comprising a nucleotide sequence complementary to the nucleotide sequence represented by SEQ ID NO: 3 or 4 and encoding a polypeptide having a sepiapterin reductase activity.
(3) A recombinant vector comprising DNA according to (2).
(4) A transformant obtained by transforming a host cell with a recombinant vector according to (3).
(5) The transformant according to (4), wherein the host is Escherichia coli.
(6) The transformant according to (4), wherein the host is Saccharomyces cerevisiae.
(7) The transformant according to (4), wherein a GTP cyclohydrolase I gene has further been transformed.
(8) The transformant according to (4), wherein a GTP cyclohydrolase I gene and a pyruvoyltetrahydropterin synthase gene have further been transformed.
(9) A method for producing biopterins, comprising: culturing a transformant according to any of (4) to (8) to produce tetrahydrobiopterin; performing oxidation treatment, if necessary; and then collecting at least one of tetrahydrobiopterin, dihydrobiopterin, and biopterin.
(10) The method for producing biopterins according to (9), wherein for the culture of the transformant, a guanine derivative or inosine derivative and a surfactant are added to the medium after a given period of time from the initiation of the culture, and the culture is further continued.
(11) The method for producing biopterins according to (10), wherein the guanine derivative is GMP (guanosine 5′-monophosphate), the inosine derivative is IMP (inosine 5′-monophosphate), and the surfactant is Triton X-100 or sodium sarcosinate.
(12) The method for producing biopterins according to (11), wherein the GMP or IMP has a concentration of 0.1 to 50 mM in the medium after the addition thereof, and the Triton X-100 or sodium sarcosinate has a concentration of 0.01 to 5% in the medium after the addition thereof.
(13) The method for producing biopterins according to (10), wherein the given period of time is 5 to 24 hours from the initiation of the culture.
(14) A method for producing biopterins, comprising using a transformant according to any of (4) to (6) to perform any reaction of the following 1) to 4):
1) a reaction through which 6-pyruvoyltetrahydropterin is used as a substrate to generate 6-1′-hydroxy-2′-oxopropyltetrahydropterin, which is further used as a substrate to generate tetrahydrobiopterin;
2) a reaction through which 6-lactoyltetrahydropterin is used as a substrate to generate tetrahydrobiopterin;
3) a reaction through which 6-1′-hydroxy-2′-oxopropyltetrahydropterin or 6-lactoyltetrahydropterin are individually used as a substrate to generate tetrahydrobiopterin by the interconversion of these two compounds or from any of these compounds; and
4) a reaction through which sepiapterin is used as a substrate to generate dihydrobiopterin.

### EFFECTS OF THE INVENTION

The use of both DNA sequences of Saccharomyces cerevisiae YIR035C-like and Bacillus subtilis yueD-like sequences found by the present inventor allows for efficient production of biopterins having effects that can be expected as pharmaceutical agents or functional foods, using microorganisms.

### BEST MODE FOR CARRYING OUT THE INVENTION

In the present specification, tetrahydrobiopterin refers to L-erythro-5,6,7,8-tetrahydrobiopterin (hereinafter, abbreviated to BH4), and the tetrahydrobiopterin and its oxidized forms L-erythro-7,8-dihydrobiopterin (hereinafter, abbreviated to BH2) and L-erythro-biopterin (hereinafter, abbreviated to biopterin) are collectively referred to as biopterins, as described in “Background Art”.

### 1. Sepiapterin Reductase

According to one aspect, the present invention provides a polypeptide of the following (A) or (B):

(A) a polypeptide comprising the amino acid sequence of SEQ ID NO: 1 or 2; and
(B) a polypeptide having an amino acid sequence derived from the amino acid sequence of the polypeptide (A) with the substitution, deletion, and/or addition of one or several amino acids and having a sepiapterin reductase activity.

“Several amino acids” are preferably 30 amino acids or less, more preferably 20 amino acids or less, even more preferably 10 amino acids or less, most preferably 9, 8, 7, 6, 5, 4, 3, or 2 amino acids or less.

According to one aspect, the present invention also provides isolated DNA of the following (a) or (b):

(a) DNA comprising the nucleotide sequence of SEQ ID NO: 3 or 4; and
(b) DNA hybridizing under stringent conditions to DNA comprising a nucleotide sequence complementary to the nucleotide sequence represented by SEQ ID NO: 3 or 4 and encoding a polypeptide having a sepiapterin reductase activity.

Hereinafter, embodiments related to SEQ ID NOS: 3 and 4 in this aspect are referred to as Saccharomyces cerevisiae YIR035C-like and Bacillus subtilis yueD-like genes, respectively.

The “sepiapterin reductase” described herein is an enzyme that catalyzes the final step of BH4 biosynthesis and has been known from a long time ago to be present mainly in mammals such as humans and rats and in birds such as chickens (Matsubara, M., et. al., Biochim. Biophys. Acta 122, 202-212 (1966)). The gene of the sepiapterin reductase has already been acquired. Specifically, the sepiapterin reductase refers to 7,8-dihydrobiopterin: NADP⁺ oxidoreductase (EC 1.1.1.153), which is an enzyme that catalyzes a reaction to generate 6-1′-hydroxy-2′-oxopropyltetrahydropterin through the action on 6-pyruvoyltetrahydropterin, a reaction to generate BH4 though the action on 6-1′-hydroxy-2′-oxopropyltetrahydropterin or 6-lactoyltetrahydropterin, and a reaction to generate BH2 through the action on sepiapterin, and can catalyze the interconversion of 6-1′-hydroxy-2′-oxopropyltetrahydropterin and 6-lactoyltetrahydropterin.

Whether or not a polypeptide encoded by a certain gene has the “sepiapterin reductase activity” may be measured directly by the assay method of Katoh et al. (Katoh, S., Arch. Biochem. Biophys., 146, 202-214 (1971)) or can be confirmed together with its in-vivo action by transforming microorganisms with the gene and confirming BH4 production in the microorganisms.

The “stringent conditions” described herein are not particularly limited as long as a nucleotide sequence contained in a test gene hybridizes to a nucleotide sequence contained in a gene having a function substantially equal to that of the test gene. Preferably, the stringent conditions are conditions involving hybridization at approximately 50° C. in a solution containing approximately 5×SSC (the composition of 1×SSC: 0.15 M NaCl, 0.015 M sodium citrate, pH 7.0), approximately 0.5% sodium dodecyl sulfate (SDS), and approximately 10 μg/ml denatured fragmented salmon sperm DNA, followed by washing at 50° C. in approximately 2×SSC and approximately 0.1% SDS.

More preferably, the stringent conditions are conditions involving hybridization under the same conditions as above, followed by washing at approximately 50° C. in approximately 1×SSC and approximately 0.1% SDS, even more preferably conditions involving hybridization under the same conditions as above, followed by washing at approximately 50° C. in approximately 0.5×SSC and approximately 0.1% SDS, still more preferably conditions involving hybridization under the same conditions as above, followed by washing at approximately 50° C. in approximately 0.1×SSC and approximately 0.1% SDS, yet more preferably conditions involving hybridization under the same conditions as above, followed by washing at approximately 65° C. in approximately 0.1×SSC and approximately 0.1% SDS.

Of course, the conditions may differ depending on DNA lengths, the sequences, and different environmental parameters. The longer the sequences are, the higher a specific hybridization temperature is. The detailed guide of nucleic acid hybridization is found in, for example, Tijssen (1993) Laboratory Techniques in Biochemistry and Molecular Biology-Hybridization with Nucleic Acid Probes part I chapter 2 “Overview of principles of hybridization and the strategy of nucleic acid probe assay” Elsevier, N.Y.

A method for obtaining the polypeptide or the DNA is not particularly limited. For example, the polypeptide or the DNA can be produced according to methods described in Examples below.

### 2. Vector

Both plasmid and phage vectors are available as a vector described herein. A preferable vector has a drug resistance gene against an appropriate antibiotic or the like as well as a restriction enzyme cleavage site for foreign gene incorporation and a promoter sequence located upstream thereof.

Examples of the vector that can be used include those commercially available from Takara Bio or Stratagene and include: versatile vectors for Escherichia coli having a promoter, such as pUC and PSTV series; and vectors for Saccharomyces cerevisiae such as pESC and pAUR series.

Preferably, the vector is selected as the vector for Escherichia coli from pUCNde (see Example 7 below), pSTVNde (see Example 7 below), and so on modified from commercially available vectors, and as the vector for Saccharomyces cerevisiae from pESC-URA (Stratagene), pESC-LEU (Strata gene), and so on, from the viewpoint of convenience of experiments.

### 3. Host and Transformant

Any host that can establish a transformation method with a vector capable of transgene incorporation and expression on the basis of techniques well known by those skilled in the art is available as a host described herein.

Examples of the host that can be used include: bacteria such as Escherichia coli, Bacillus subtilis, Corynebacterium, and Rhizobium; actinomyces such as Streptomyces; yeasts such as Saccharomyces cerevisiae, Schizosaccharomyces pombe, and Pichia pastoris; molds such as Aspergillus; animal cells; and plant cells.

Microorganisms such as bacteria and yeasts are advantageous, and, preferably, the host is selected from E. coli, S. cerevisiae, and so on, from the viewpoint of practical utility in substance production.

Both DNAs of Saccharomyces cerevisiae YIR035C-like and Bacillus subtilis yueD-like genes found by the present inventor can be ligated with the appropriate plasmid vector, with which a host such as Saccharomyces cerevisiae or Escherichia coli is transformed to thereby obtain a transformant.

### 4. Culture

The transformant can be cultured in a medium containing necessary nutrients to thereby produce biopterins.

After the completion of culture, biopterins are secreted in the culture solution. Alternatively, the biopterins when aerobically cultured are sometimes present in the form of BH2 or biopterin in the medium because a portion or most of biosynthesized BH4 undergoes oxidation. Thus, the produced BH4 and its oxidized form BH2 may be subjected to oxidation treatment, if necessary, from the viewpoint of simplifying the step of collecting the biopterins (particularly biopterin) from the culture solution. For example, a chemical method or a biochemical method using enzymes or the like may be performed as the oxidation treatment described herein. The method is not particularly limited as long as it does not degrade biopterins. A convenient method is a chemical method using an oxidizer, which can be performed, for example, by adding an oxidizer such as potassium iodide to the culture solution or a processed product thereof.

### 5. Collection of Biopterins

Biopterins produced into the culture solution can be purified after collection by a purification method or the like known in the art to thereby obtain desired kinds of biopterins (e.g., BH4, BH2, or biopterin).

The collection step described herein involves initially separating microbial cells from the culture solution by a method such as filtration or centrifugation. Biopterins may be extracted from the cells by physically destructing the cells by use of ultrasonication, a Braun homogenizer (Braun, Melsungen, Germany)), or the like or can be extracted advantageously by, for example, the extraction method of Kohashi et al. (Kohashi, et al. Agric. Biol. Chem., 44, 2089-2094 (1980)) wherein the cells are suspended in 0.1 N hydrochloric acid and then treated at 120° C. for 2 minutes. The biopterins can be isolated from the culture supernatant or the cell extracts by using carriers alone or in combination appropriate for column chromatography, such as active carbon, Florisil, alumina, silicate, powdered filter papers, porous synthetic polymer carriers (e.g., Amberlite XAD-2 (Rohm and Haas) and Diaion (Mitsubishi Chemical)), a variety of ion-exchange resins (e.g., DOWEX 50WX8 (Acros Organics), Amberlite IRC-50 (Rohm and Haas), DEAE Sepharose CL-6B (Amersham Biosciences)), gel filtration carriers (e.g., Sephadex G-25 (Amersham Biosciences), Bio-Gel P-2 (Bio-Rad)), and a variety of affinity carriers.

### 6. BH4 Biosynthesis Enzyme Genes (GCHI and PTPS)

Of three enzyme genes involved in BH4 biosynthesis, not only the SPR (sepiapterin reductase) gene but also GCHI (GTP cyclohydrolase I) and/or PTPS (pyruvoyltetrahydropterin synthase) genes are ligated to the plasmid vector. As a result, these two or three genes can be introduced simultaneously into a host to thereby significantly increase the production of biopterins. In addition, two or three species of plasmids comprising one or more of these three enzyme genes may be introduced into a host.

These biosynthesis enzyme genes such as GCHI and PTPS can be obtained by PCR reaction using their respective chromosomal DNAs on the basis of DNA sequence information on the genes generally known by those skilled in the art and a database thereof known in the art, as described in Examples below. Preferably, PCR primers are designed to give sequences comprising appropriate restriction enzyme cleavage sites added to both terminal portions of the genes, and PCR reaction using the primers produces the genes that are easy to insert into a vector.

To express these biosynthesis enzyme genes, the vector described in the paragraph “Vector” and the gene are cleaved with appropriate restriction enzymes and ligated with the biosynthesis enzyme genes cleaved with the same enzymes as those used for the cleavage of the vector. Then, a host such as Escherichia coli or Saccharomyces cerevisiae can be transformed with the vector to thereby prepare a production strain.

In the culture of the recombinant production strain, a 2YT medium, L medium, or the like is available for Escherichia coli, while an S medium (8 g of glucose, 13 g of (NH₄)₂SO₄, 7.9 g of NaH₂PO₄.2H₂O, 3 g of polypeptone, 3 g of yeast extract, 2 g of KCl, 0.8 g of MgSO₄.7H₂O, 0.1 g of NaCl, 90 mg of FeSO₄.7H₂O, 60 mg of ZnSO₄.7H₂O, 10 mg of MnSO₄.4-6H₂O, 5 mg of CuSO₄.5H₂O, dissolved in 1 L of deionized water (hereinafter, abbreviated to “/1 L”, pH 7.0), SD-UraLeu medium (20 g of glucose, 1.7 g of Yeast nitrogen base (without amino acid/(NH₄)₂SO₄), 5 g of (NH₄)₂SO₄, 20 mg of adenine sulfate, 20 mg of Arg, 100 mg of Asp, 100 mg of Glu, 30 mg of Ile, 30 mg of Lys, 20 mg of Met, 50 mg of Phe, 400 mg of Ser, 200 mg of Thr, 30 mg of Tyr, 150 mg of Val, 20 mg of His, 20 mg of Trp/1 L), or the like can be used for Saccharomyces cerevisiae. Biopterins can be separated from the culture solution after culture.

The gene that can be used for advantageously producing biopterins using microorganisms, the microorganism introducing the gene therein, and the production of biopterins using the microorganism have been described above with reference to the embodiments of the present invention.

### EXAMPLES

Hereinafter, the present invention will be described more specifically with reference to Examples. However, the present invention is not intended to be limited to these Examples. Basic procedures related to gene recombination below were performed using methods described in, for example, Molecular Cloning, A Laboratory Manual, Third Edition, Cold Spring Harbor Laboratory Press (2001), Methods in Enzymology, vol. 194 (1991), Text for Bioengineering Experiments (Seibutsu Kogaku Jikkensho in Japanese), revised edition, Baifukan Co., Ltd. (2002), Experimental Medicine (Jikken Igaku in Japanese), special edition, Gene Experiments Using Yeasts, Yodosha Co., Ltd. (1994). Commercially available kits were used according to the instructions included therein.

### Example 1

### YIR035C Sequence and yueD Sequence

BH4 production by certain species of microorganisms among molds and yeasts has been revealed by Shiraishi et al. (Patent Documents 1 and 2). The full genomic sequence of Saccharomyces cerevisiae has already been determined. Therefore, the full genomic sequence obtained from a database was initially used to search for sequences highly homologous to known SPR sequences derived from a variety of organisms (these sequences are available from, e.g., KEGG (Kyoto Encyclopedia of Genes and Genomes) Database (see URL: http://www.genome.ad.jp/kegg/) of Kyoto University) by use of sequence comparison software Blast. As a result, two highly homologous sequences YIR035C and YIR036C were found from the search based on human and mouse SPR sequences.

Of them, it has already been reported as to YIR036C that a protein expressed therefrom has no confirmed SPR activity (Maruyama, R. et al., J. Biotechnol, 94, 157-169 (2002)). On the other hand, it was found that studies such as functional prediction were not conducted on the DNA sequence fragment of YIR035C. Thus, whether YIR035C exhibits a function as SPR was studied. SEQ ID NO: 3 of sequence listing represents the nucleotide sequence of a YIR035C gene from Saccharomyces cerevisiae, and SEQ ID NO: 1 of sequence listing represents an amino acid sequence encoded by the gene.

This YIR035C gene has homology as quite low as 28% and 26% to the human and mouse SPR sequences, respectively, and has merely been described as “ORF, Uncharacterized, Hypothetical protein” even in the database (http://db.yeastgenome.org/) that discloses the contents of the yeast genome project. Moreover, whether or not Saccharomyces cerevisiae, the origin of the YIR035C gene, generally has a biopterin biosynthesis pathway is also unknown. No documents except for Patent Documents 1 and 2 have been found to mention this point. Furthermore, those skilled in that art generally expect that a protein expressed from the YIR035C gene has no SPR activity, from the information of the document stating that YIR036C highly homologous to the YIR035C gene has no confirmed SPR activity. On these technical assumptions, the present inventor of the present application has found for the first time that a protein expressed from the YIR035C gene unexpectedly has an SPR activity, as described later.

Many SPR-like sequences whose expressed proteins have no confirmed SPR activity have been known. A yueD sequence derived from Bacillus subtilis, one of the SPR-like sequences, was studied as to whether it exhibits a function as SPR, as with YIR035C. SEQ ID NO: 4 represents the nucleotide sequence of a yueD gene from Bacillus subtilis, and SEQ ID NO: 2 represents an amino acid sequence encoded by the gene.

### Example 2

### Acquisition of Saccharomyces cerevisiae and Bacillus subtilis SPR-Like Sequences and Preparation of their Respective Expression Plasmids for Saccharomyces cerevisiae

First, the genomic DNA of a Saccharomyces cerevisiae NBRC10102 strain was extracted using GenTorukun (Takara Bio). Then, a sense primer YIR035C-F (SEQ ID NO: 5: cggaattcatgggtaaagttattttagttacagg) and an antisense primer YIR035C-R (SEQ ID NO: 6: cgggatccctcaaggcataaagtccgccaaggc) were designed as PCR primers for Saccharomyces cerevisiae YIR035C sequence acquisition using a PCR method. PCR was performed using the genomic DNA as a template and these two PCR primers to thereby obtain a YIR035C gene having cleavage sites for restriction enzymes EcoRI and BamHI in 5′- and 3′-noncoding regions, respectively.

This PCR product was digested with EcoRI and BamHI and inserted into the EcoRI and BglII sites of a pESC-URA vector (Stratagene) to prepare a plasmid pEU (GAL10-YIR035C) having a Saccharomyces cerevisiae SPR-like sequence.

A DNA sequencer was used to confirm that this plasmid comprised the DNA sequence represented by SEQ ID NO: 3.

Next, genomic DNA was extracted from a Bacillus subtilis ATCC14593 strain using GenTorukun. PCR was performed using the genomic DNA and a sense primer yueD-F (SEQ ID NO: 7: cggaattcatggaactttatatcatcaccggagc) and an antisense primer yueD-R (SEQ ID NO: 8: cgggatccctacaaaaactctttaatatcataaatgcgg) designed for Bacillus subtilis yueD sequence acquisition using a PCR method to thereby obtain a yueD gene having cleavage sites for restriction enzymes EcoRI and BamHI in 5′- and 3′-noncoding regions, respectively.

This PCR product was digested with EcoRI and BamHI and inserted into the EcoRI and BglII sites of a pESC-URA vector (Stratagene) to prepare a plasmid pEU (GAL10-yueD) having a Bacillus subtilis SPR-like sequence. A DNA sequencer was used to confirm that this plasmid comprised the DNA sequence represented by SEQ ID NO: 4.

### Example 3

### Acquisition of Biopterin Biosynthesis-System Gene and Preparation of Coexpression Plasmid for Saccharomyces cerevisiae

PCR was performed using the genomic DNA of the Bacillus subtilis ATCC14593 strain as a template and a sense primer mtrA-F (SEQ ID NO: 9: cgggatccatatgaaagaagttaataaagagcaaatcg) and an antisense primer mtrA-R (SEQ ID NO: 10: ccgctcgagttagtcctggcgtttaatatgttcc) designed for Bacillus subtilis mtrA sequence (GTP cyclohydrolase I gene derived from Bacillus subtilis) acquisition using a PCR method. This PCR product was digested with BamHI and XhoI and inserted into the BamHI and XhoI sites of a pESC-URA vector (Stratagene) to prepare a plasmid pEU (GAL1-mtrA).

This fragment digested with BamHI and XhoI was inserted into the BamHI and XhoI sites of each of the plasmids pEU (GAL10-YIR035C) and pEU (GAL10-yueD) to prepare plasmids pEU (GAL1-mtrA/GAL10-YIR035C) and pEU (GAL1-mtrA/GAL10-yueD).

Subsequently, PCR was performed using a rat cDNA library (Stratagene) as a template and a sense primer rPTPS-F (SEQ ID NO: 11: ggaattccatatgaacgcggcggttggccttcggcgc) and an antisense primer rPTPS-R (SEQ ID NO: 12: gaagatctctattctcctttgtagaccacaatgttgttg) designed for rat PTPS sequence acquisition using a PCR method.

This PCR product was digested with EcoRI and BglII and inserted into the EcoRI and BglII sites of a pBluescriptII KS (−) vector (Stratagene). A fragment obtained by digestion at SalI and XbaI sites was inserted into the SalI and NheI sites of a pESC-LEU vector (Stratagene) to prepare a plasmid pEL (GAL1-ptps).

### Example 4

### Acquisition of Transformed Saccharomyces cerevisiae

Saccharomyces cerevisiae YPH499 strains [mat A, ura3, leu2, trp1, his3, ade2, lys2] (Stratagene) were transformed with the following plasmids prepared in Example 3:

(1) plasmids pEU (GAL1-mtrA/GAL10-YIR035C) (see Example 3) and pEL (GAL1-ptps) (see Example 3);
(2) plasmids pEU (GAL1-mtrA/GAL10-yueD) and pEL (GAL1-ptps);
(3) plasmids pEU (GAL1-mtrA) and pEL(GAL1-ptps); and
(4) plasmids pESC-URA and pESC-LEU,
to obtain (1) Y4-mp5, (2) Y4-mpy, (3) Y4-mp, and (4) Y4-EL strains, respectively.

Transformants were obtained using FastTrack-Yeast Transformation Kit (Takara Bio) and selected on an SD-UraLeu agar medium (20 g of glucose, 1.7 g of Yeast nitrogen base (without amino acid/ammonium sulfate), 5 g of ammonium sulfate, 20 mg of adenine sulfate, 20 mg of Arg, 100 mg of Asp, 100 mg of Glu, 30 mg of Ile, 30 mg of Lys, 20 mg of Met, 50 mg of Phe, 400 mg of Ser, 200 mg of Thr, 30 mg of Tyr, 150 mg of Val, 20 mg of His, 20 mg of Trp/1 L, 2% agar was added to agar medium).

### Example 5

### Production of Biopterins by Transformed Saccharomyces cerevisiae

The transformed Saccharomyces cerevisiae obtained in Example 4 was shake-cultured at 30° C. for 24 hours in 5 ml of modified SD-UraLeu medium (20 g of glucose, 13.6 g of Yeast nitrogen base (without amino acid/ammonium sulfate), 5 g of ammonium sulfate, 160 mg of adenine sulfate, 160 mg of Arg, 800 mg of Asp, 800 mg of Glu, 240 mg of Ile, 240 mg of Lys, 160 mg of Met, 400 mg of Phe, 3200 mg of Ser, 1600 mg of Thr, 240 mg of Tyr, 1200 mg of Val, 160 mg of His, 160 mg of Trp/1 L), and the culture solution was inoculated at 5% into 2 ml of SG-UraLeu medium (80 g of galactose, 13.6 g of Yeast nitrogen base (without amino acid/ammonium sulfate), 5 g of ammonium sulfate, 160 mg of adenine sulfate, 160 mg of Arg, 800 mg of Asp, 800 mg of Glu, 240 mg of Ile, 240 mg of Lys, 160 mg of Met, 400 mg of Phe, 3200 mg of Ser, 1600 mg of Thr, 240 mg of Tyr, 1200 mg of Val, 160 mg of His, 160 mg of Trp/1 L) and further cultured for 96 hours. A BP (biopterins) content in the supernatant of the resulting culture solution was measured.

The measurement of the BP content was conducted by analysis using liquid chromatography (column: Lichrospher RP-18 5 μm, φ4×250 mm; eluant: 5% methanol, 40 mM citric acid, 20 mM KH₂PO₄ (pH 3.0); flow rate: 1 ml/min.; detection: fluorescence at 450 nm excited at 350 nm). As a result, it was revealed that the Y4-mp 5 strains produced 20 μg/ml biopterin, the Y4-mpy strains produced 15 μg/ml biopterin, the Y4-mp strains produced 0.2 μg/ml biopterin, and the Y4-EL strains produced 0.01 μg/ml biopterin in the culture solutions. When compared with Saccharomyces cerevisiae introduced only a vector therein and Saccharomyces cerevisiae introduced mtrA and a rat PTPS gene therein, Saccharomyces cerevisiae introduced therein either a YIR035C or yueD gene in addition to mtrA and a rat PTPS gene was evidently improve in productivity. The use of a YIR035C or yueD gene as an SPR gene exhibited higher productivity and smaller variations in productivity among transformations than the use of a rat SPR gene shown in Reference Example 2.

### Example 6

### Production of Biopterins by Transformed Saccharomyces cerevisiae (Culture Involving Addition)

The obtained transformed Saccharomyces cerevisiae Y4-mp 5 strains were shake-cultured at 30° C. for 24 hours in 5 ml of modified SD-UraLeu medium in the same way as in Example 5, and the culture solution was inoculated at 5% into 2 ml of modified SG-UraLeu medium. After 8 hours from the initiation of the culture, 2 mg of GMP and 0.1 ml of 2% Triton X-100 were added thereto, and the cells were further cultured for 96 hours. A BP content in the supernatant of the resulting culture solution was measured. As a result, the biopterins content in the culture supernatant was 37 μg/ml, indicating evident increases in yield as compared with a culture supernatant unsupplemented with guanine and Triton X-100.

In Examples, “GMP (preferably, added at a concentration of 0.1 to 50 mM in the medium after the addition thereof)” is illustrated as an example of a “guanine derivative”. However, the guanine derivative is not limited to this, and guanine, guanosine, GDP, or GTP may also be used. In Examples, “Triton X-100” is illustrated as an example of a “surfactant”. However, the surfactant is not limited to this, and “sodium sarcosinate (preferably, added at a concentration of 0.01 to 5% in the medium after the addition thereof, as with the “Triton X-100)” may also be used.

In Examples, “after 8 hours from the initiation of the culture” is illustrated as timing of addition of the “guanine derivative” and the “surfactant”. However, the timing is not limited to this, and the range of “after 5 to 24 hours from the initiation of the culture” is also adoptable.

One of causes of the increases in yield resulting from the addition of the guanine derivative and the surfactant as described above may be that the addition of the surfactant increases the permeability of the cell membrane and cell wall of the microorganism, and the guanine derivative is thus taken up into the cell and utilized in the biosynthesis of biopterins.

### Example 7

### Production of Biopterins by Transformed Escherichia coli

The YIR035C fragment obtained in Example 2 was cleaved with restriction enzymes NdeI and BamHI and ligated with a plasmid pSTVNde (plasmid in which the NdeI cleavage site of pSTV29 (Takara Bio) was deleted by the blunting of the sticky end, and an NdeI cleavage site was constructed at the initiation codon portion of a lacZ gene) cleaved with NdeI and BamHI, with which Escherichia coli DH5α (Toyobo Co., Ltd.) was transformed. This strain was spread on a 2YT plate supplemented with IPTG, X-Gal, and chloramphenicol and cultured at 37° C. White colonies grown therefrom were selected to obtain a plasmid pSTVYIR035C of interest.

The PTPS fragment obtained in Example 3 was cleaved at both terminal portions with restriction enzymes NdeI and BglII (Takara Bio) and ligated with a plasmid pUCNde (plasmid in which the NdeI cleavage site of pUC19 was deleted by the blunting of the sticky end, and an NdeI cleavage site was constructed at the initiation codon portion of a lacZ gene) cleaved with NdeI and BamHI, with which Escherichia coli DH5α (Toyobo Co., Ltd.) was transformed. This strain was spread on a 2YT plate supplemented with IPTG, X-Gal, and ampicillin and cultured at 37° C. White colonies grown therefrom were selected to obtain a plasmid pUCPTPS of interest.

The recombinant Escherichia coli having this pUCPTPS was treated with calcium chloride according to a standard method and then transformed with the pSTVYIR035C. The strain was spread on a 2YT plate supplemented with ampicillin and chloramphenicol and cultured at 37° C. Colonies grown therefrom were selected. The recombinant Escherichia coli having both the pSTVYIR035C and the pUCPTPS was inoculated into a test tube medium (24 mm in diameter) containing 5 ml of N medium (20 g of glycerol, 10 g of casamino acid, 4 g of yeast extract, 4 g of K₂HPO₄, 4 g of KH₂PO₄, 2.7 g of NaHPO₄.2H₂O, 2 g of MgSO₄.7H₂O, 1.2 g of (NH₄)₂SO₄, 0.2 g of NH₄Cl, 40 mg of FeSO₄.7H₂O, 40 mg of CaCl₂.2H₂O, 10 mg of MnSO₄.5H₂O, 10 mg of AlCl₃.6H₂O, 4 mg of CoCl₂.6H₂O, 2 mg of ZnSO₄.7H₂O, 2 mg of Na₂MoO₄.2H₂O, 1 mg of CuCl₂.2H₂O, 0.5 mg of H₃BO₃, dissolved in 1 L of deionized water (pH 7.0)) supplemented with 50 mM IPTG, 50 μg/ml ampicillin, and 20 μg/ml chloramphenicol in terms of the final concentrations. Then, the cells were shake-cultured at 37° C. for 8 hours. Then, 2 mg of GMP (guanosine 5′-monophosphate) as a guanine derivative and 0.1 ml of 2% Triton X-100 as a surfactant were added thereto, and the cells were further cultured for 48 hours. After culture, the culture solution was centrifuged, and the resulting supernatant was analyzed by liquid chromatography. As a result, a biopterin peak was obtained, and a yield thereof was 1.0 μg per ml of the medium.

### Reference Example 1

### Acquisition of Rat SPR Gene and Preparation of Coexpression Plasmid for Saccharomyces cerevisiae

PCR was performed using Bacillus subtilis genomic DNA as a template and a sense primer mtr-F2 (SEQ ID NO: 13: cggaattcatgaaagaagttaataaagagcaaatcg) and an antisense primer mtr-R2 (SEQ ID NO: 14: cgggatccttagtcctggcgtttaatatgttcc) designed for Bacillus subtilis mtrA sequence acquisition using a PCR method. This PCR product was digested with EcoRI and BamHI and inserted into the EcoRI and BglII sites of a pESC-URA vector to prepare a plasmid pEU (GAL10-mtrA).

Furthermore, PCR was performed using a rat cDNA library (Stratagene) as a template and a sense primer rSPR-F (SEQ ID NO: 15: cgggatcccatatggaaggaggcaggctaggttgcgctg) and an antisense primer rSPR-R (SEQ ID NO: 16: ccgctcgagttaaatgtcatagaagtccacgtgggc). This PCR product was digested with BamHI and XhoI and inserted into the BamHI and XhoI sites of the pEU (GAL10-mtrA) to prepare a plasmid pEU (GAL1-spr/GAL10-mtrA). A DNA sequencer was used to confirm that this plasmid comprised the DNA sequence represented by SEQ ID NO: 18. An amino acid sequence encoded by the DNA represented by SEQ ID NO: 18 is shown in SEQ ID NO: 17.

### Reference Example 2

### Production of Biopterins by Rat SPR-Coexpressing Saccharomyces cerevisiae

Saccharomyces cerevisiae YPH499 strains were transformed with the plasmid pEU (GAL1-spr/GAL10-mtrA) prepared in Reference Example 1 and the plasmid pEL (GAL1-ptps) to prepare Y4-mps strains. The obtained transformed Saccharomyces cerevisiae was shake-cultured at 30° C. for 24 hours in 5 ml of modified SD-UraLeu medium, and the culture solution was at 5% inoculated into 2 ml of SG-UraLeu medium and further cultured for 96 hours. A BP content in the supernatant of the resulting culture solution was measured. As a result, it was revealed that the cells produced up to 4 μg/ml biopterins in the culture solution. However, great variations in yield were observed among the transformants.

On the other hand, large yields of biopterins as well as small variations in yield among transformants and stable production of biopterins can be confirmed in the cells of Example 5. From these results together, it has been deduced that in Saccharomyces cerevisiae, a rat SPR gene sequence may cause a gene to be instable and easily deleted, mRNA to be instable, and gene expression efficiency to vary depending on subtle environmental influence.

1. A polypeptide of the following (A) or (B):
(A) a polypeptide comprising the amino acid sequence of SEQ ID NO: 1 or 2; and
(B) a polypeptide having an amino acid sequence derived from the amino acid sequence of the polypeptide (A) with the substitution, deletion, and/or addition of one or several amino acids and having a sepiapterin reductase activity.

2. Isolated DNA of the following (a) or (b):
(a) DNA comprising the nucleotide sequence of SEQ ID NO: 3 or 4; and
(b) DNA hybridizing under stringent conditions to DNA comprising a nucleotide sequence complementary to the nucleotide sequence represented by SEQ ID NO: 3 or 4 and encoding a polypeptide having a sepiapterin reductase activity.

3. A recombinant vector comprising DNA according to claim 2.

4. A transformant obtained by transforming a host cell with a recombinant vector according to claim 3.

5. The transformant according to claim 4, wherein the host is Escherichia coli.

6. The transformant according to claim 4, wherein the host is Saccharomyces cerevisiae.

7. The transformant according to claim 4, wherein a GTP cyclohydrolase I gene has further been transformed.

8. The transformant according to claim 4, wherein a GTP cyclohydrolase I gene and a pyruvoyltetrahydropterin synthase gene have further been transformed.

9. A method for producing biopterins, comprising:
culturing a transformant according to any of claims 4 to 8 to produce tetrahydrobiopterin; performing oxidation treatment, if necessary; and then collecting at least one of tetrahydrobiopterin, dihydrobiopterin, and biopterin.

10. The method for producing biopterins according to claim 9, wherein for the culture of the transformant, a guanine derivative or inosine derivative and a surfactant are added to the medium after a given period of time from the initiation of the culture, and the culture is further continued.

11. The method for producing biopterins according to claim 10, wherein the guanine derivative is GMP (guanosine 5′-monophosphate), the inosine derivative is IMP (inosine 5′-monophosphate), and the surfactant is Triton X-100 or sodium sarcosinate.

12. The method for producing biopterins according to claim 11, wherein the GMP or IMP has a concentration of 0.1 to 50 mM in the medium after the addition thereof, and the Triton X-100 or sodium sarcosinate has a concentration of 0.01 to 5% in the medium after the addition thereof.

13. The method for producing biopterins according to claim 10, wherein the given period of time is 5 to 24 hours from the initiation of the culture.

14. A method for producing biopterins, comprising using a transformant according to any of claims 4 to 8 to perform any reaction of the following (1) to (4):
(1) a reaction through which 6-pyruvoyltetrahydropterin is used as a substrate to generate 6-1′-hydroxy-2′-oxopropyltetrahydropterin, which is further used as a substrate to generate tetrahydrobiopterin;
(2) a reaction through which 6-lactoyltetrahydropterin is used as a substrate to generate tetrahydrobiopterin;
(3) a reaction through which 6-1′-hydroxy-2′-oxopropyltetrahydropterin and 6-lactoyltetrahydropterin are separately used as a substrate to generate tetrahydrobiopterin by the interconversion of these two compounds or from any of these compounds; and
(4) a reaction through which sepiapterin is used as a substrate to generate dihydrobiopterin.


SEQUENCE LISTING (18 sequences)

SEQ ID NO: 1 - protein, 254 aa, Saccharomyces cerevisiae
MGKVILVTGVSRGIGKSIVDVLFSLDKDTVVYGVARSEAPLKKLKEKYGDRFFYVVGDITEDSVLKQLVNAAVKGHGKIDSLVANAGVLEPVQNVNEIDVNAWKKLYDINFFSIVSLVGIALPELKKTNGNVVFVSSDACNMYFSSWGAYGSSKAALNHFAMTLANEERQVKAIAVAPGIVDTDMQVNIRENVGPSSMSAEQLKMFRGLKENNQLLDSSVPATVYAKLALHGIPDGVNGQYLSYNDPALADFMP

SEQ ID NO: 2 - protein, 243 aa, Bacillus subtilis
MELYIITGASKGLGQAIALQALEKGHEVHALSRTKTDVSHKKLTQHQIDLINLEEAEQQFETLLSSIDSDRYSGITLINNAGMVTPIKRAGEASLDELQRHYQLNLTAPVLLSQLFTKRFASYSGKKTVVNITSGAAKNPYKGWSAYCSSKAGLDMFTRTFGFEQEDEELPVNMISFSPGVMDTEMQAVIRSSSKKDFHHIERFRKLNETGSLRSPDFIAGTLLSLLEKGTENGRIYDIKEFL

SEQ ID NO: 3 - DNA, 765 bp, Saccharomyces cerevisiae
atgggtaaagttattttagttacaggtgtttccagaggtatcggtaagtccatcgtggatgttcttttcagtttggacaaggacacggttgtttacggtgtagccaggtctgaggcacccttgaagaagttgaaagagaagtatggcgacaggtttttttacgttgtcggtgatattaccgaggattccgtgttgaagcagttggttaacgctgctgttaagggccacggcaagatcgactccttggttgccaacgctggtgtcctagagcccgtgcaaaatgtcaacgagattgatgtcaacgcttggaagaagctgtatgacatcaacttcttcagcattgtttccttggttggcattgcgttacctgaattgaagaagaccaacggtaacgtggtattcgtcagttcggacgcctgtaacatgtacttcagcagttggggagcttacggttcttcaaaagccgctctgaaccacttcgccatgactctggccaacgaggaaaggcaagtgaaagccattgccgtcgccccaggtattgtggacacagatatgcaagttaacattagggagaacgtggggccttcctccatgagtgcagagcaattgaagatgtttagaggtttaaaggagaataaccagttgctggatagctctgtgccagctacagtttatgccaaattggcccttcatggtattcctgacggtgttaatggacagtacttgagctataatgaccctgccttggcggactttatgccttga

SEQ ID NO: 4 - DNA, 732 bp, Bacillus subtilis
atggaactttatatcatcaccggagcgtcaaaagggctgggtcaagccattgcattacaggctttagaaaaggggcatgaagtccatgccttatccagaacgaaaacagatgtctctcacaaaaaactaacgcagcatcaaatagacctcatcaatctcgaagaagctgaacagcaatttgaaacattgctctcatccatcgattcagatcgttattctggtattacccttattaataacgccggaatggtaacgccgatcaaacgtgccggcgaagcgtctcttgacgagcttcagcgccattatcagctgaacctgactgcgcccgtgcttttgagtcagctgtttacaaaacggtttgcttcatacagcggcaaaaagacggttgtcaacattacttcaggagccgccaaaaatccatataagggatggagcgcgtattgcagttcaaaagccgggctcgacatgtttacgaggacattcggatttgaacaggaggatgaagagctgccggtgaacatgatttcgttctcacctggagtgatggacactgagatgcaggccgtcatccgttcttcatcgaaaaaggatttccaccacattgaacgattccggaaattaaatgaaacaggaagccttcgcagtccggactttattgccggcacgctgctttctttactagaaaaagggacggaaaacggccgcatttatgatattaaagagtttttgtag

SEQ ID NO: 5 - DNA, 34 bp, Artificial Sequence
  Synthetic primer YIR035C-F
cggaattcatgggtaaagttattttagttacagg

SEQ ID NO: 6 - DNA, 33 bp, Artificial Sequence
  Synthetic primer YIR035C-R
cgggatccctcaaggcataaagtccgccaaggc

SEQ ID NO: 7 - DNA, 34 bp, Artificial Sequence
  Synthetic primer yueD-F
cggaattcatggaactttatatcatcaccggagc

SEQ ID NO: 8 - DNA, 39 bp, Artificial Sequence
  Synthetic primer yueD-R
cgggatccctacaaaaactctttaatatcataaatgcgg

SEQ ID NO: 9 - DNA, 38 bp, Artificial Sequence
  Synthetic primer mtrA-F
cgggatccatatgaaagaagttaataaagagcaaatcg

SEQ ID NO: 10 - DNA, 34 bp, Artificial Sequence
  Synthetic primer mtrA-R
ccgctcgagttagtcctggcgtttaatatgttcc

SEQ ID NO: 11 - DNA, 37 bp, Artificial Sequence
  Synthetic primer rPTPS-F
ggaattccatatgaacgcggcggttggccttcggcgc

SEQ ID NO: 12 - DNA, 39 bp, Artificial Sequence
  Synthetic primer rPTPS-R
gaagatctctattctcctttgtagaccacaatgttgttg

SEQ ID NO: 13 - DNA, 36 bp, Artificial Sequence
  Synthetic primer mtrA-F2
cggaattcatgaaagaagttaataaagagcaaatcg

SEQ ID NO: 14 - DNA, 33 bp, Artificial Sequence
  Synthetic primer mtrA-R2
cgggatccttagtcctggcgtttaatatgttcc

SEQ ID NO: 15 - DNA, 39 bp, Artificial Sequence
  Synthetic primer rSPR-F
cgggatcccatatggaaggaggcaggctaggttgcgctg

SEQ ID NO: 16 - DNA, 36 bp, Artificial Sequence
  Synthetic primer rSPR-R
ccgctcgagttaaatgtcatagaagtccacgtgggc

SEQ ID NO: 17 - protein, 262 aa, Rattus norvegicus
MEGGRLGCAVCVLTGASRGFGRALAPQLAGLLSPGSVLLLSARSDSMLRQLKEELCTQQPGLQVVLAAADLGTESGVQQLLSAVRELPRPERLQRLLLINNAGTLGDVSKGFLNINDLAEVNNYWALNLTSMLCLTTGTLNAFSNSPGLSKTVVNISSLCALQPFKGWGLYCAGKAARDMLYQVLAVEEPSVRVLSYAPGPLDTNMQQLARETSMDPELRSRLQKLNSEGELVDCGTSAQKLLSLLQRDTFQSGAHVDFYDI

SEQ ID NO: 18 - DNA, 789 bp, Rattus norvegicus
atggaaggaggcaggctaggttgcgctgtctgcgtgctgaccggggcttcccggggcttcggccgcgccctggccccgcagctggccgggttgctgtcgcccggttcggtgttgcttctaagcgcacgcagtgactcgatgctgcggcaactgaaggaggagctctgtacgcagcagccgggcctgcaagtggtgctggcagccgccgatttgggcaccgagtccggcgtgcaacagttgctgagcgcggtgcgcgagctccctaggcccgagaggctgcagcgcctcctgctcatcaacaatgcaggcactcttggggatgtttccaaaggcttcctgaacatcaatgacctagctgaggtgaacaactactgggccctgaacctaacctccatgctctgcttgaccaccggcaccttgaatgccttctccaatagccctggcctgagcaagactgtagttaacatctcatctctgtgtgccctgcagcccttcaagggctggggactctactgtgcagggaaggctgcccgagacatgttataccaggtcctggctgttgaggaacccagtgtgagggtgctgagctatgccccaggtcccctggacaccaacatgcagcagttggcccgggaaacctccatggacccagagttgaggagcagactgcagaagttgaattctgagggggagctggtggactgtgggacttcagcccagaaactgctgagcttgctgcaaagggacaccttccaatctggagcccacgtggacttctatgacatttaa